(19) United States
(12) Patent Application Publication
(10) Pub. No.: US 2016/0143583 A1
(43) Pub. Date: May 26, 2016

(54) Hydration Monitoring System

(71) Applicant: PepsiCo, Inc., Purchase, NY (US)

(72) Inventors: Asker JEUKENDRUP, Purchase, NY (US); Mark KOVACS, Purchase, NY (US); Andrew ANDERSON, Purchase, NY (US); James CARTER, Purchase, NY (US); Ian ROLLO, Purchase, NY (US); Gregory YEP, Purchase, NY (US); Andrew LINTOTT, Cambridge (GB); Nicola MILLAR, Cambridge (GB); Robert MILNER, Cambridge (GB)

(21) Appl. No.: 14/866,497

(22) Filed: Sep. 25, 2015

Related U.S. Application Data
(63) Continuation-in-part of application No. PCT/US2015/020972, filed on Mar. 17, 2015.
(60) Provisional application No. 61/969,427, filed on Mar. 24, 2014.

(51) Int. Cl. A61B 5/00; G01F 1/68; G01F 23/00; G01F 1/10; A61B 5/01; A61B 5/11

(52) U.S. Cl. CPC A61B 5/4875; A61B 5/486; A61B 5/7445; A61B 5/4266; A61B 5/01; A61B 5/112; A61B 5/6801; A61B 5/6887; A61B 5/0002; G01F 23/00; G01F 1/10; G01F 1/68; A61B 2562/0247

(57) ABSTRACT

A hydration monitoring system for the collection of data about fluid consumption and hydration levels of athletes during training or practice sessions. The system may also measure and analyze carbohydrate consumption. The system utilizes a hydration bottle containing a fluid and to measures the amount of fluid consumed in a given time interval and wirelessly transmit the measurements; and a scale configured to measure the weight of an athlete and wirelessly transmit the measurements. The system further utilizes a data communications hub configured to receive data comprising the measurements from the hydration bottle and scale and forward the data to a computer; and a computer configured to receive the data from the hub for analysis. The computer analyzes the data and calculates whether the athlete should consume more or less fluid and/or more or less carbohydrates.

### CROSS-REFERENCE

This is a continuation-in-part of PCT Application No. PCT/US2015/020972 filed Mar. 17, 2015, which claims the benefit of U.S. provisional application No. 61/969,427, filed Mar. 24, 2015. Each of these applications is incorporated herein by reference in its entirety.

### BACKGROUND

### Field of the Invention

The invention relates to a system for monitoring hydration of an athlete, in particular the invention relates to a hydration monitoring system for the collection of data about fluid consumption and hydration level of athletes during training or practice sessions. The system may also be used to monitor other parameters such as carbohydrates and electrolytes.

### BRIEF SUMMARY

Proper hydration aids an athlete in obtaining optimal performance during training and athletic events such as, for example, basketball, hockey, or track. Monitoring hydration and the effect of hydration on athletic performance is an advancing field of science. It is particularly desirable to monitor and study hydration levels in athletes while they train, for example by monitoring their fluid consumption and fluid loss. One system to monitor hydration manually records player weights and fluid levels in the athlete's bottles and then analyzes the results. This system is laborious and can only reveal hydration levels in post-session analysis.

The need to monitor hydration in real time has only recently been proposed but the means to do so have not been practical. The industry has looked toward wireless technologies; however until recently, none have been suitable because A) such technologies were not implemented in commonly available portable computers and B) the technology has not been available on microchips with a sufficiently low power demand and small physical size so as to enable integration into a drink bottle, for example.

It is therefore desired to obtain an effective system of monitoring fluid consumption and fluid loss and providing analysis in real time. Such monitoring method must not significantly interfere with the conduct of the training session and results should be available during the training session so that immediate action can be taken based on the analysis.

A first aspect of the invention is directed to a hydration monitoring system for evaluating hydration of an athlete. The system utilizes a hydration bottle containing a fluid, wherein the bottle is configured to measure the amount of fluid consumed in a given time interval and wirelessly transmit the measurements; a scale, wherein the scale is configured to measure the weight of the athlete and wirelessly transmit the measurements; a data communications hub, wherein the hub is configured to receive data comprising the measurements from the hydration bottle and scale and forward the data to a computer; and a computer configured to receive the data from the hub for analysis, wherein the computer analyzes the data and calculates whether the athlete should consume more or less fluid; and a display for displaying the results of the measurements and analysis. The system may further be used to monitor and display other parameters such as carbohydrates and/or electrolytes and analyze collected data and determine whether the athlete should consume more or less carbohydrate(s) and/or electrolytes.

A further aspect of the invention is directed to a method of monitoring hydration of an athlete comprising: measuring an amount of fluid consumed by an athlete from a hydration bottle containing fluid and periodically transmitting the measurements to a data communications hub; measuring the weight of an athlete and transmitting the measurements to the data communications hub; forwarding measurements collected by the data communications hub to a computer; and analyzing the measurements and calculating whether the athlete should consume more fluid; and displaying the results of the measurements and analysis.

Another aspect of the invention is directed to a hydration bottle for measuring fluid consumption comprising a bottle having a removable cap assembly, the cap assembly having an opening for dispensing fluid, a flow meter positioned within the cap assembly below the opening, electronics to record flow measurements from the flow meter, and a transceiver to transmit the measurements to a data communications hub.

Another aspect of the invention is directed to a hydration monitoring system comprising a bottle, wherein the bottle is configured to measure an amount of fluid consumed by an athlete and wirelessly transmit data corresponding to the measurement, a fluid-loss device, wherein the fluid-loss device is configured to determine an amount of fluid lost by the athlete and wirelessly transmit data corresponding to the amount, a data communications hub, wherein the hub is configured to receive data from the bottle and the fluid-loss device, a computer, wherein the computer is configured to receive the data from the hub and determine whether the athlete should consume more fluid based on the data; and a display for displaying an output conveying the data and the determination.

Another aspect of the invention is directed to a method of monitoring hydration of an athlete, the method comprising measuring an amount of fluid consumed by an athlete from a bottle and periodically transmitting data corresponding to the measurement to a data communications hub, determining an amount of fluid lost by the athlete using a fluid-loss device and transmitting data corresponding to the amount to the data communications hub, forwarding the data collected by the data communications hub to a computer, and determining whether the athlete should consume more fluid based on an analysis of the data; and displaying the results of the determination on a display.

### BRIEF DESCRIPTION OF THE DRAWINGS

FIG. 1A illustrates a wireless-enabled hydration measurement architecture that may be utilized in accordance with an aspect of the disclosure.

FIG. 1B illustrates devices useful in the hydration measurement architecture of FIG. 1A.

FIG. 2 illustrates a bottle containing a flow measurement device in accordance with an aspect of the disclosure.

FIG. 3 illustrates an exploded view of the cap of the bottle of FIG. 2.

FIG. 4 illustrates a turbine style flow meter used in the cap of the present invention.

FIG. 5 illustrates the cap assembly of FIG. 2 containing a flow meter in accordance with at least one aspect of the invention.

FIG. 6 illustrates a top side of a circular disk containing electronics useful with the flow measurement device.

FIG. 7 illustrates a bottom side of a circular disk containing electronics useful with the flow measurement device.

FIG. 8A and FIG. 8B illustrate two aspects of thermal flow meters useful in the present invention.

FIG. 9 illustrates a bottle containing an LED display in accordance with an aspect of the invention.

FIG. 10 illustrates an exploded view of the bottle of FIG. 9.

FIG. 11 illustrates of an exploded view of an alternative embodiment of the bottle of FIG. 2, the bottle containing an insert.

FIG. 12A illustrates an insert in accordance with FIG. 10 containing a flow measurement device without the battery cover in place and FIG. 12B illustrates the insert of FIG. 11 with the battery cover in place.

FIG. 13 illustrates a case containing a scale in accordance with an aspect of the invention.

FIGS. 14A-14G illustrate various screen shots of a tablet utilizing the hydration monitoring system of the present invention.

### DETAILED DESCRIPTION OF THE INVENTION

Aspects of the present invention address the need for real-time analysis to allow real-time adjustment of an athlete's hydration program during a training session. A wireless-enabled monitoring system enables collection and recordation of data pertaining to fluid consumption and weight of one or more athletes. The present invention integrates fluid and weight measurement devices into an integrated system allowing information from those measurements to be analyzed. The integrated system of measurement devices transmits data wirelessly to a computer that is capable of performing data analytics and displaying analyzed results.

In accordance with aspects of the invention, bottles containing fluid have flow measurement devices incorporated therein to measure the amount of fluid consumed and electronics to record the measurements and time. In addition, fluid-loss devices are used to determine the amount of fluid that is lost by the athlete. For example, in some embodiments, scales are used to measure the weight of athletes and to record the times the measurements are taken. In some embodiments, a sweat patch may be used to measure the amount of fluid lost by the athlete. Other methods of measuring the amount of fluid lost by the athlete are also envisioned. The measured data including recorded times are then ultimately transmitted from the bottles and scales to a computer for analysis. The data is then analyzed on the computer and displayed on the computer's screen. The measurements and analysis may all occur in real-time.

The system may further be used to monitor and display other parameters such as carbohydrates and/or electrolytes and analyze collected data and determine whether the athlete should consume more or less carbohydrate(s) and/or electrolytes. For ease of discussion, the application will be described in terms of hydration and fluid consumed. However, measurement of other parameters is also contemplated.

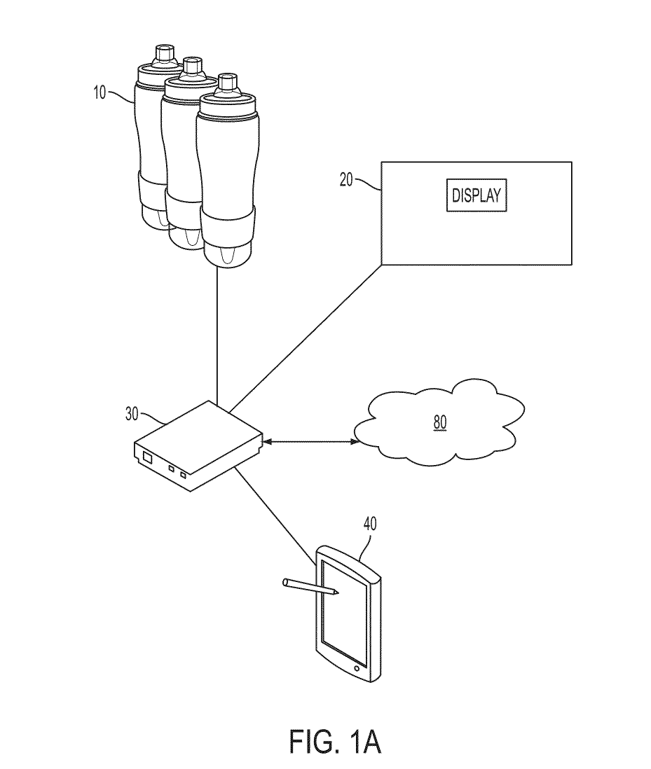

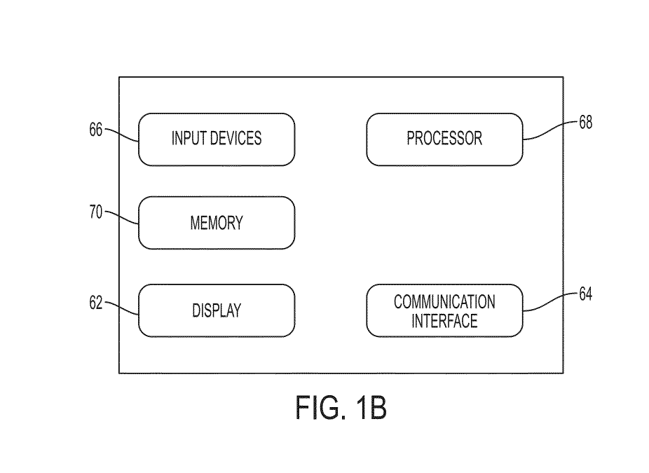

As shown in FIG. 1A, a hydration monitoring system utilizes one or more drink bottle(s) (10), one or more scale(s) (20) to measure and record weight, one or more data communications hub(s) (30), and a computer (40). Each of the devices features Bluetooth

Smart transceivers that enable collected fluid consumption, weight, and time data from the bottles and scales to be transmitted via the hub to the computer where the data is recorded, analyzed, and displayed. Bluetooth Smart transceivers, for example, are particularly desirable as they have low power consumption, small physical size, and have recently become available on a range of mobile computing devices. Other suitable transceivers or transmitters may be used with the present system.

As further illustrated in FIG. 1A, the devices and machines described above may be operatively connected to each other through a communications network, such as communications network (80).

As illustrated in FIG. 1B, the various devices shown in FIG. 1A (10, 20, 30, and 40) may each comprise a memory (66), a processor (70), a display (72) (which may include touchscreens), and a communication interface (74). Each processor (70) may execute computer-executable instructions present in memory (66) such that, for example, the devices may send and receive information to and from each other directly or through network (80).

The devices in FIG. 1B may also include various input devices (76). The input devices may include keyboards, track balls, mice, joy sticks, buttons, and readers.

In one aspect of the invention, the communications interfaces (64) and/or devices may be networked together through communications network (80). Communications network (80) may represent: 1) a local area network (LAN); 2) a simple point-to-point network (such as direct modem-to-modem connection); and/or 3) a wide area network (WAN), including the Internet and other commercial based network services. In one aspect, the interfaces and/or devices may be connected to each other through communications network (80) using various well-known protocols, such as TCP/IP, Ethernet, FTP, HTTP, BLUETOOTH, Wi-Fi, ultra wide band (UWB), low power radio frequency (LPRF), radio frequency identification (RFID), infrared communication, IrDA, third-generation (3G) cellular data communications, Global System for Mobile communications (GSM), or other wireless communication networks or the like may be used as the communications protocol. The interfaces and/or devices may be physically connected to each other or one or more networks via twisted pair wires, coaxial cable, fiber optics, radio waves or other media.

The term “network” as used herein and depicted in the drawings should be broadly interpreted to include not only systems in which remote storage devices are coupled together via one or more communication paths, but also stand-alone devices that may be coupled, from time to time, to such systems that have storage capability. Consequently, the term “network” includes not only a “physical network” but also a “content network,” which is comprised of the data—attributable to a single entity—which resides across all physical networks. A “network,” as used herein, may also include a network of “virtual” servers, processes, threads, or other ongoing computational processes which communicate with each other, some or all of which may be hosted on a single machine which may provide information to client servers, processes, threads or other ongoing computational processes on that same machine, other game machines, or both.

### Bottle

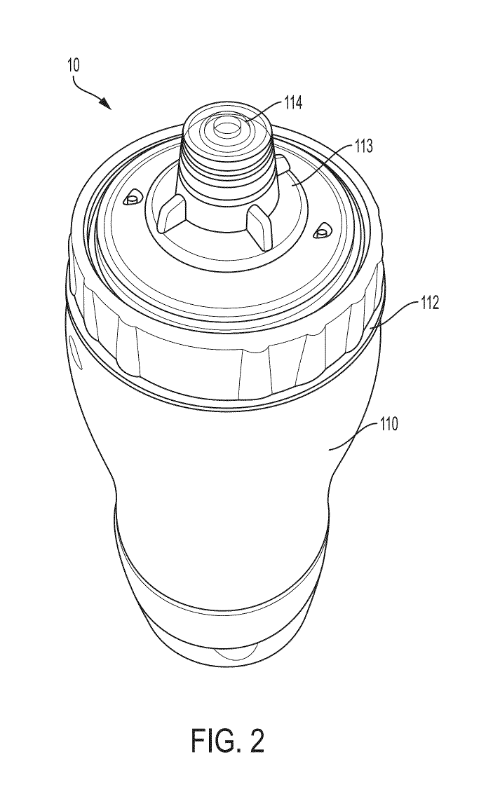

FIG. 2 depicts an example of a bottle (10) that could be used with the present invention. The bottle may have stiff (nonflexible) walls or squeezable (flexible) walls. Generally the bottle is made of a plastic (polymeric) material. A squeezable bottle allows fluid to be forced out at a faster rate than a bottle with stiff (inflexible) side walls. In a particular aspect, the bottle has squeezable walls.

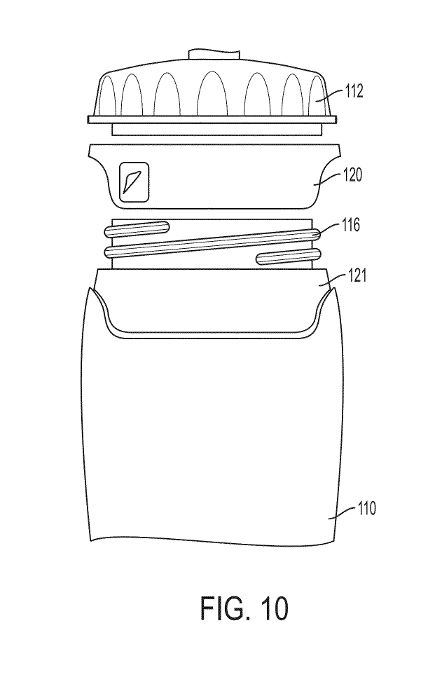

The bottle has a base (110) to hold a fluid and a cap assembly (112) having a neck (113) and an opening (114) above the neck for dispensing the fluid. The cap assembly (112) typically is attached to the bottle through a threaded connection (116) as best shown in FIG. 10 although snap-type or other connections are also possible. The athlete picks up and tilts the bottle and the squeezes the bottle to allow/force the fluid therein to flow out.

Each of the bottles contains a flow measurement device (flow sensor or flow meter) and electronics that measure the volume of fluid dispensed and store that information until it is wirelessly transmitted to the data communications hub. In particular, the bottles contain a flow measurement device for measuring the volume of fluid consumed, electronics, a power source, and a Bluetooth Smart transceiver (or other suitable transceiver or transmitter) for transmitting the fluid measurements and time of consumption to one or more data communication hubs. The bottles may store data in internal memory until transmitted to a hub. The data may also be reflected in a bottle mounted display.

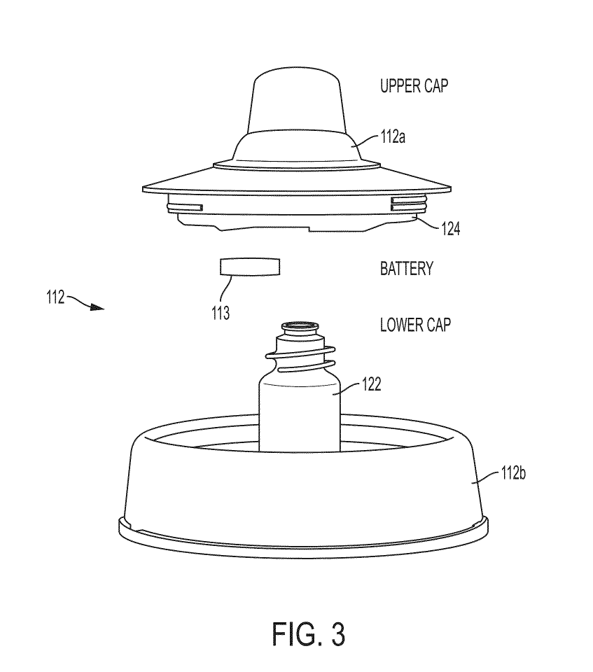

FIG. 3 illustrates an exploded view of cap assembly (112) having an upper cap (112a) and lower cap (112b). Upper cap (112a) is received by lower cap (112b) and the upper and lower caps may be attached together via screw threads or snap fit. In this aspect, flow measurement device (122) is positioned in lower cap (112b) and electronics (124) are positioned in upper cap (112a). The electronics include a transceiver (or transmitter) device, e.g. a Bluetooth Smart transceiver, and a receptacle (not shown in this FIG.) for a battery (113). The electronics including transceiver are powered by the battery. Any suitable battery may be used such as a coin style lithium battery (113) or a permanent rechargeable battery which is recharged via metal contacts made external to the electronics enclosure. In one aspect, the battery level may be checked from the computer.

It is important to obtain reliable fluid volume measurements from the flow measurement device (122) integrated into the bottle. Many commercially available flow meters have poor accuracy or are too large for the required space envelope or are not responsive to non-steady flows.

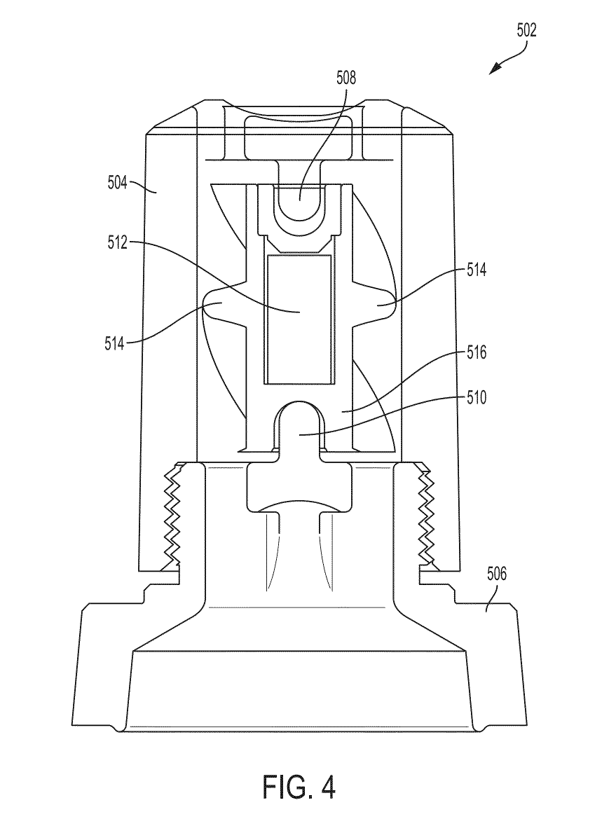

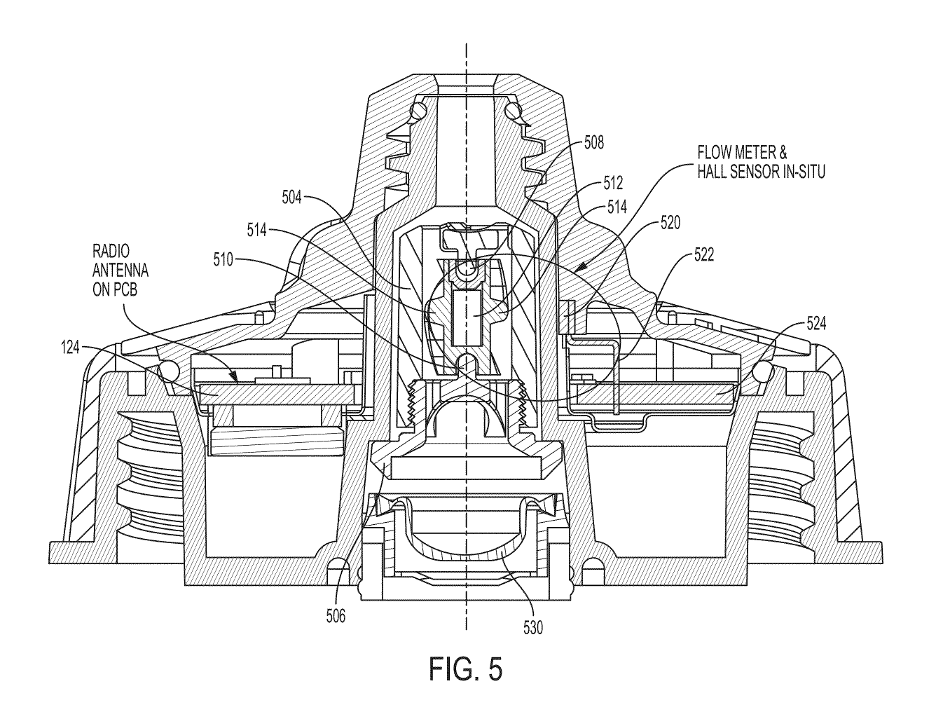

As shown in FIG. 4, a turbine flow meter (502) may be used to measure volume flow in accordance with an aspect of the disclosure. Turbine flow meter (502) may be an axial screw style turbine meter based on the Archimedes screw. In an embodiment, turbine flow meter (502) may include a two part housing (504) and (506). Each part of the two part housing (504) and (506) may be positioned within (or connected to) a hemispherical spigot (508) and (510) in order to provide consistent resistance to motion in all orientations. For instance, turbine flow meter (502) may be centrally located or positioned in a cap assembly (112) as illustrated in FIG. 3. The hemispherical spigots (508) and (510) may keep the two part housing (504) and (506) aligned and correctly positioned as cap assembly (112) is repositioned, reoriented, or tilted during use. In an embodiment, turbine flow meter (502) may include a diametrically polarized magnet (512) as illustrated in FIG. 5A. The diametrically polarized magnet (512) may be operatively coupled to a Hall Effect switch sensor (520) (shown in FIG. 5). In an embodiment, the rate of spin turbine flow meter (502) will be proportional to the velocity of the flow. The Hall Effect switch sensor (520) may produce an alternating digital output twice per rotation of turbine flow meter (502).

Turbine flow meter (502) may include a number of blades (514) positioned along a rotor (516) of turbine flow meter (502). The blades (514) may be pitched to optimize fluid flow and fluid flow detection. In addition, the ratio of inner diameter to the outer diameter and the overall diameter of the turbine flow meter (502) may also be optimized based on criteria such as 1) lowest flow rate at which turbine meter (505) will rotate in water, 2) highest rotation rate at moderate and high flow rates, and 3) lowest volumetric measurement variance in pulsatile dispense tests. In an alternative embodiment, blades (514) may include the diametrically polarized magnet.

FIG. 5 shows placement of turbine flow meter (502) in cap assembly (112). In one embodiment, turbine flow meter (502) may be positioned in series with an elastomeric valve (530) which has the property of withholding fluid flow until a sufficient back pressure has been generated, then releasing fluid freely. The elastomer valve (530) may assist in overcoming static friction in the turbine mounting and establish free running of the turbine flow meter (502). A circular disk (524) surrounds the flow meter and contains electronics (124), e.g. a printed circuit board (“PCB”). A Hall Sensor is connected to the circular disk via wire (522.)

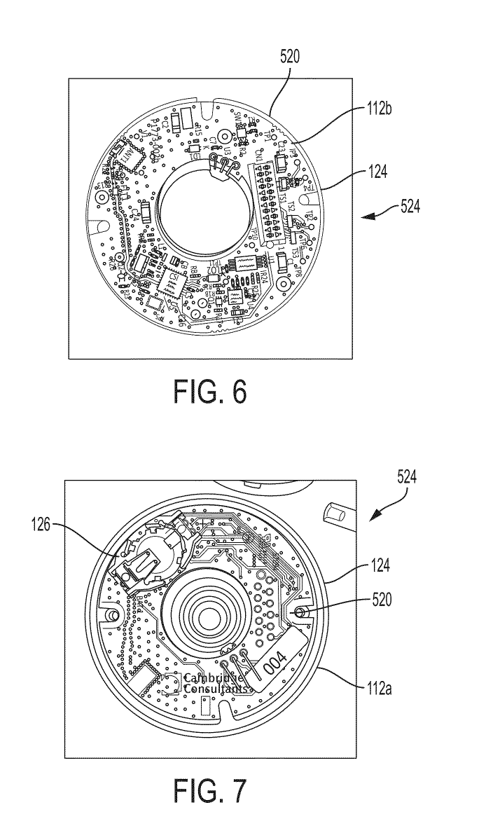

FIG. 6 illustrates the top side of the circular disk (524) positioned in lower cap (112b). FIG. 7 illustrates the bottom side of a circular disk (524) positioned in upper cap (112a). Circular disk (524) contains electronics (124). A receptacle (126) holds a battery (not shown). In this aspect, the electronics include the memory to store flow measurements and transceiver to transmit the measurements.

Other types of flow measurement devices or mass flow meters useful in the present invention are “thermal mass flow meters.” In addition, a circular disk is exemplified as holding the electronics. Other designs and shapes of platforms may be utilized to hold the electronics or PCB.

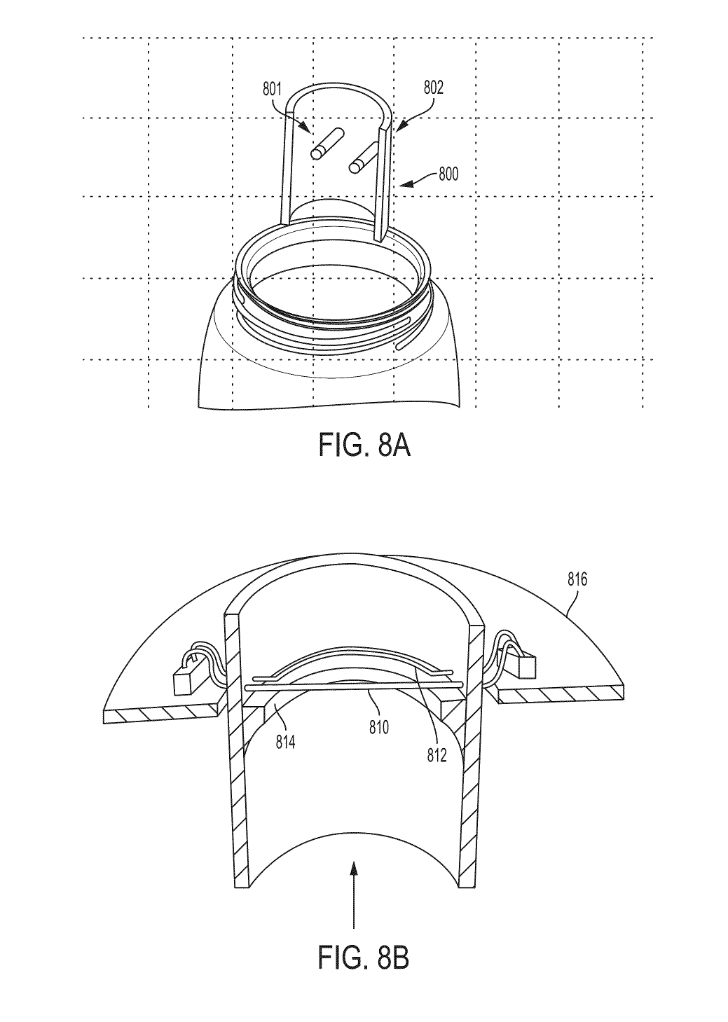

FIG. 8a is directed to a thermal mass flow meter in accordance with one aspect of the invention. The thermal mass flow meter has two parallel wires arranged to cross the flow path. In one embodiment both wires are hollow thermistors (801) (802). One thermistor (801) contains a resistive heating element to maintain a temperature different between the thermistors. The PCB drives the heating element and monitors the wire temperature signal using a PID (Proportional, Integral, Derivative) control algorithm to maintain a constant temperature difference between the wires. When fluid flows, heat is transferred from the heated wire causing it to cool. The PID algorithm supplies current to the wire to maintain the temperature difference and the energy supplied is related to the mass flow of fluid past the wire by a deterministic equation. The unheated wire (802) will come to the temperature of the fluid.

FIG. 8b is directed to a thermal mass flow meter in accordance with another aspect of the invention. The two wires (810) and (812) consist of fine strands of resistive material such as ni-chrome. One wire (812) is shielded from most of the flow by a feature (814) in the flow path, while the other (810) is exposed to the full flow of the fluid. A constant current flows in the wires and the resistance of the wires is monitored by the PCB (816). When fluid flows, the wires will cool differentially and the measured resistance will change. The shielded wire (812) gives a base line measurement sensitive to the temperature of the fluid, the other wire (810) will lose heat to the fluid and is sensitive to both the fluid temperature and the flow rate.

To achieve low power usage in either aspect, the PCB features an accelerometer which is used to detect the orientation of the bottle and heat the wires when the bottle is tilted. The wires are as fine a possible to minimize their heat capacity and the resistance detection is done with high sensitivity to allow a minimal driving current to be used. Sufficient power must be supplied to heat the wires.

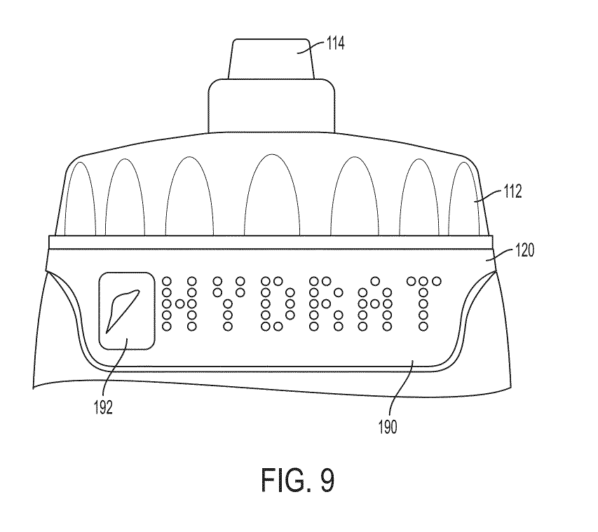

As shown in FIG. 9, the bottles would feature an electronic display (190) as part of interface necker (120). For example, the display is a matrix of LEDs. This display may show a variety of information including the amount of fluid consumed. The display may be the result of data analysis occurring elsewhere in the system such as real time feedback from the computer. For example, for coaching applications, the data may indicate that the user should drink more based on the calculated hydration deficit. The display may use words, numbers, or colors. The display may be a text stream such as notes from the coach. The display may indicate increased hydration is necessary or encouraged. A more sophisticated system may display how much more fluid should be consumed. A button (192) may be present to turn on and off the display or to cycle the display content amongst information stored internally in the display component.

FIG. 10 illustrates an exploded view of FIG. 9. An interface necker (120), fitted into a recess (121) on base (110) and positioned beneath the cap assembly (112), may be present to convey information to the athlete or coach. For example, an LED display may be part of interface necker (120). In one aspect, the LED display has its own battery and Bluetooth Low Energy receiver.

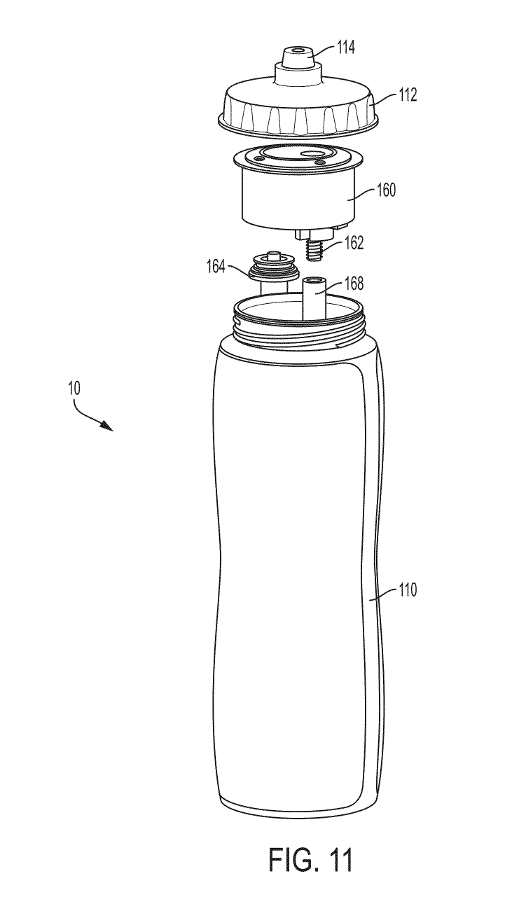

In an alternative aspect, as shown in FIG. 11 an insert (160) sits between base (110) and cap assembly (112). The insert (160) is held in place by the cap assembly (112), which attaches to base (110) trapping the insert in position. A straw (168) may be attached to the insert through straw receiver (162).

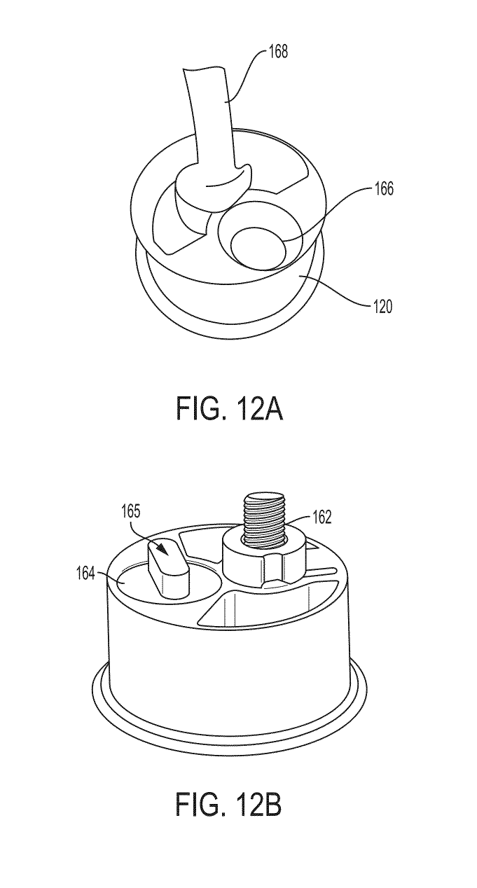

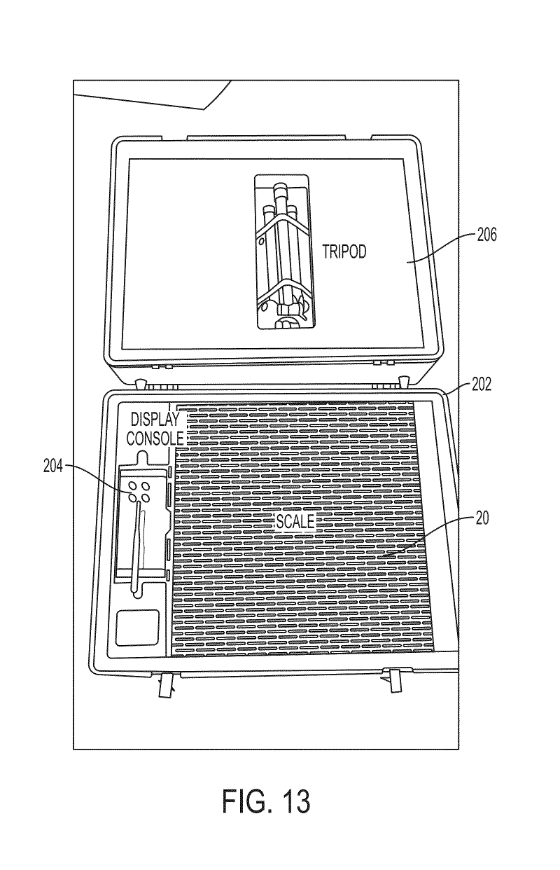

As shown in FIG. 12A and FIG. 12B, a battery would be positioned in cavity (166) and battery cover (164) screwed into place via threads. The battery cover may have a tab (165) to aid in removing and replacing the battery cover (164). Insert (160) houses the flow measurement device, electronics, a power source (battery), and a transceiver or transmitter device.

### Fluid-Loss Devices

The scale(s) (20) measure and record the weight of the athletes and may be an off-the-shelf product. The scale surface may be modified by the application of a non-slip surface such as a treaded rubber mat.

In one aspect the scale is housed in an enclosure (202) designed to give the scale stability when placed on the ground in the training area. The enclosure (202) may be an off-the-shelf case and customized to protect the scale. The enclosure (202) may have a pair of skids (not shown) to increase the contact area of the unit with the ground and stiffen the case, particularly where the scale is used on an uneven surface such as a grassy field.

The enclosure (202) may house various other components including the scales display (204) and one or more tripods (202). Other components (not shown) that may be stored in the case are battery charger(s) and one or more data communications hubs (30). The tripods may be any suitable tripods suitable to hold the display (202) and/or to mount the data communications hubs (30). The scales may have any suitable power source, but typically have batteries such as built-in lead-acid rechargeable batteries, which must be periodically recharged.

Display (204) displays the weight of the athlete and may be modified to house a Bluetooth Smart transceiver for transmitting weight measurements and the time of the measurement to the data communications hubs (30). Although less desired, a manual reading may be taken of the weights and inputted into the computer directly.

The fluid-loss device of the system may be something other than a scale. In some embodiments the fluid-loss device may be a sensor. The sensor may be worn by the athlete, for example, as clothing or a patch adhered to the skin. In some embodiments where the sensor is a patch, the sensor may have a porous membrane configured to adhere to the athlete's skin, a microfluidics layer in contact with the porous membrane, a sensor module fluidly connected to the porous membrane, a memory, an antenna, and an outer textile layer. In some embodiments, the microfluidics layer transports the fluid from the porous membrane to the sensor module, and the sensor determines a change of an ion concentration present in the fluid. Some examples of these types of sensors are described in U.S. Pat. No. 7,383,072, U.S. Publication No. 2015/0112165, U.S. Publication No. 2013/0197319, and U.S. Publication No. 2011/0152643. These references are incorporated by reference in their entirety. In some embodiments, the sensor may include a layer with a characteristic that changes with the amount of fluid that the layer comes into contact with. For example, the impedance of the layer may increase or decrease with the amount of fluid. Some examples of this type of fluid-loss device are described in U.S. Pat. No. 8,057,454 and U.S. Publication No. 2011/0152718. These references are incorporated by reference in their entirety.

In some embodiments, the fluid-loss device may be comprised of sensors that are worn by the athlete. An example of a fluid-loss device using a sensor that may be incorporated into clothing is described in U.S. Pat. No. 8,306,599B2, which is incorporated by reference in its entirety. In some embodiments, the fluid-loss device may measure temperature, humidity, or pressure which may then be used to calculate fluid lost of the athlete. Some examples of these types of fluid-loss devices are described in U.S. Pat. No. 8,306,599 and U.S. Pat. No. 5,131,390. These references are incorporated by reference in their entirety. In some embodiments the fluid-loss device may comprise different types of sensors that are carried by the athlete during their athletic activity, including but not limited to a pedometer, a temperature sensor, a pressure sensor, moisture or humidity sensor. An example of this type of fluid-loss device is described in U.S. Pat. No. 7,493,232, the entirety of which is incorporated by reference. In some embodiments the fluid-loss device may comprise a user input and a processor that calculates the amount of fluid loss of the athlete based on a value inputted by the athlete. An example of a type of this fluid-loss device is described in U.S. Pat. No. 6,138,079A, the entirety of which is incorporated by reference.

### Communication Hubs

One or more data communication hubs (30) collect and forward data to a data recording and display device or computer (40). The data communication hub may be mounted on a tri-pod (206). The hub may be custom built based on commonly available chip sets, for example, Bluetooth Smart chips, such as for example CSR1010 devices. In one aspect, the hub contains two Bluetooth Smart transceivers, which utilize the Bluetooth Smart chips. One transceiver receives data from a multitude of devices and the other transceiver maintains a persistent link to the computer whenever it is in radio range. The hub further comprises batteries, for example 4 AA batteries.

The data communication hubs should be elevated for better communication with the bottles, scales, and computer. In one aspect multiple hubs are used to improve radio coverage within a single venue.

In another aspect, multiple hubs are used when athletes are training in multiple locations. A hub is assigned to an area comprising one or more locations. The hubs are then capable of transmitting to a centrally located computer or the hubs may communicate between themselves in order to synchronize a global data model, effectively increasing the overall coverage of the radio system. Alternatively, the computer (e.g. a tablet style device) may travel to (be carried to) the hub locations to wirelessly connect with each hub to download information.

A communications hub need not be utilized and instead the present system may utilize the Bluetooth capabilities of the portable computer. However, it has been found that manufacturer's implementation of the Bluetooth Smart connection layer in portable computers can render the system vulnerable to software bugs. To avoid such problems, the separate communication hub was created to reduce reliance on the portable computer.

### Computer

The recording and display device or computer (40) may be any suitable computer such as a laptop. In a particular aspect, a tablet such as an iPad® is used. The program may scan for wireless signals from the hubs. Once contact is made, the data transmitted is stored in a log file in the computer.

The computer (40) stores a time referenced record of the fluid and weight measurements, and performs data analysis on those measurements to provide real-time or near real-time information, for example on a graphical user interface. Any suitable software and programs may be used to collect and process the data from the bottles and scales. Such program may be in the form of an App which is downloaded by the user onto a tablet.

The wireless communications architecture of the present invention enables near real-time collection of data applied to hydration monitoring. In a particular aspect, the wireless communication uses Bluetooth Smart transceivers in advertising mode to send small data packets without establishing a full Bluetooth Smart connection. Under certain conditions the system can establish a Bluetooth Smart connection, but this may be performed optimally to minimize the number of simultaneous connections that must be maintained. Thus the advertising mode cuts down on the overhead of data transmission.

The system allows for integrated and expandable system of devices sharing the same Bluetooth broadcast architecture. The system provides reliable data collection and communications architecture robust to potential loss of radio signal. Loss of data would lead to incorrect results and would render the remainder of the data unusable. The multiplexed connections of multiple transceivers to a single hub (or small number of hubs) are in a scalable way that minimizes the number of Bluetooth Smart connections that must be made. The analysis of collected data in real time provides immediate feedback to athletes and coaches.

### Computer Operation

The system is set up with the computer, bottles, scale, and communication hub. Personal data for each athlete is inputted into the computer. Such data may be, but not limited to, name, position, date of birth, age, height, and a photograph.

The computer display may have main views and pop up views and may be customized depending on the sport, number of sessions, number of athletes, and the like. A session may be set up by recording an athlete's name and/or ID number, the bottle number assigned to the athlete, weight and/or body mass, and the type of fluid the athlete will be consuming. This step is repeated for each athlete. Session notes may be added in a notes section, for example, if an athlete is not feeling well or if the athlete has taken medication.

A device view may display the connection status for each bottle including battery level, calibration factors, and bottle associations. For instance, calibration factors for the turbine flow meter include constants which correspond to the slope and y-axis intercept of a linear best fit of calibration measurements. The calibration measurements relate the volume of fluid dispensed to the number of turbine rotations measured. The Flow/Rev and the Flow Offset (angle of bottle when the athlete is drinking) may also be displayed. The settings of the bottle may be edited, for example, drink timeouts (ms) and impeller timeouts (ms) may be edited. A typical value for each may be 3000 ms. The drink timeout refers to the time after a sip to determine that the drink is complete. A drink consists of multiple sips which are added together and reported as a single drink.

A detailed view may be provided for each athlete to display details for each athlete such as age, height, sport, position, initial body mass, nude body mass pre and post, and fluid being consumed and, if relevant, carbohydrates or electrolytes consumed.

An athlete's data may be displayed on a popup which displays weight and fluid readings for each athlete and times each measurement was made. The popup may reflect change in body mass, sweating rate, and fluid consumed.

The calorific content of the fluid is recorded and used to calculate carbohydrate consumption. Other nutritional information (sodium intake, for example) may be recorded as well for various calculations.

The formulae used to determine the athlete's hydration level is based on the weight deficit and the amount of fluid consumed. The equations are:

Body weight change=Current body weight−Starting body weight

Cumulative sweat mass=Mass of fluid consumed−Body weight change

Sweat mass delta=Current body weight−Previous measured body weight+Mass of fluid consumed in-between weight measurements.

### System Operation

Each athlete is associated with the bottle they are using, for example by assigning the athlete and bottle the same number. A fluid is selected for consumption by the athlete. The fluid may be a hydration fluid such as water or solutions containing electrolytes and/or carbohydrates such as GATORADE®. The fluid may be prepared with water and powder.

Bottles are filled with the selected fluid by unscrewing the cap and removing the insert. Then the selected fluid is added to the bottle, the insert replaced, and the cap screwed on.

The communication hub is mounted on a tripod. The scale is set up and prepared for use. The initial clothed weight of each athlete is measured and recorded. Any other data relevant to calculate hydration is recorded as well as any other data one wishes to monitor.

The athlete begins the training and periodically takes drinks from the assigned bottle. A “drink” is considered to be composed of several individual sips of fluid over a period of time referred to as the “drink timeout”. After drinking, the bottles wait for this time period to ensure that the drink is complete before registering the volume of fluid consumed. It is assumed that the amount consumed is the amount dispensed by the bottle.

The bottles communicate with the hub whenever an athlete uses a bottle. This may not occur immediately if the athlete is out of range. In one aspect, the bottle is capable of storing a large number of measurements. Thus, if the bottle is not in range of the communications hub when the measurements are taken (generally between 10-20 meters), then at the end of a session (or intermittently during the session), the bottle can be moved near the communications hub to transmit the stored measurements.

During or after a session, the data and details can be checked on the computer. A final check that all data has been collected may be made by having the athlete take a final drink. Data should arrive at the computer within a set period of time, for example, 15 s, following the end of the drink timeout.

After the training session is over, the final clothed weight is measured and recorded for each athlete. A nude body mass (post) weigh reading is taken for each athlete. The computer then analyzes and displays the results.

The following table represents exemplary measurements for a possible session using a turbine flow meter:

| | Athlete | | | | | | | | |
|---|---|---|---|---|---|---|---|---|---|
| Bottle | | | | Pre-Nude | Post Nude | | | Bottle | Bottle |
| ID | age | height | Sport | Weight | Weight | Drink-type | Carbs | Slope | Offset |
| 1 | 23 | 192 | darts | 67.75 | | Gatorade | 14 | 0.529 | 0.628 |
| 2 | 0 | 0 | basketball | 80.95 | | Gatorade | 14 | 0.624 | 0.276 |
| 3 | 37 | 193 | basketball | 76.75 | | Gatorade | 14 | 0.597 | −0.092 |
| 4 | 20 | 183.62 | baseball | 80.2 | | Gatorade | 14 | 0.657 | −1.917 |
| 5 | 20 | 201.31 | baseball | 78.42 | | Gatorade | 14 | 0.685 | −1.677 |
| 6 | 20 | 211 | basketball | 69.5 | | G2 | 5 | 0.578 | −0.501 |
| 7 | 0 | 0 | basketball | 75.45 | | G2 | 5 | 0.47 | 4.887 |
| 8 | 18 | 188 | basketball | 89.42 | | Endurance | 14 | 0.498 | 2.806 |
| | | | | | | Formula | | | |
| 9 | 17 | 199.7 | basketball | 73.81 | | Endurance | 14 | 0.612 | 1.372 |
| | | | | | | Formula | | | |
| 10 | 20 | 204.2 | basketball | | | Water | 0 | 0.481 | 3.539 |
| 11 | 19 | 200.4 | basketball | 82.5 | | Water | 0 | 0.56 | 0.338 |
| 13 | 24 | 208.4 | basketball | | | Water | 0 | 0.542 | 2.441 |

The following are representative of views from a hydration monitoring session. The possible views are not limited to the following and the views may be tailored for individuals, sports, and the like.

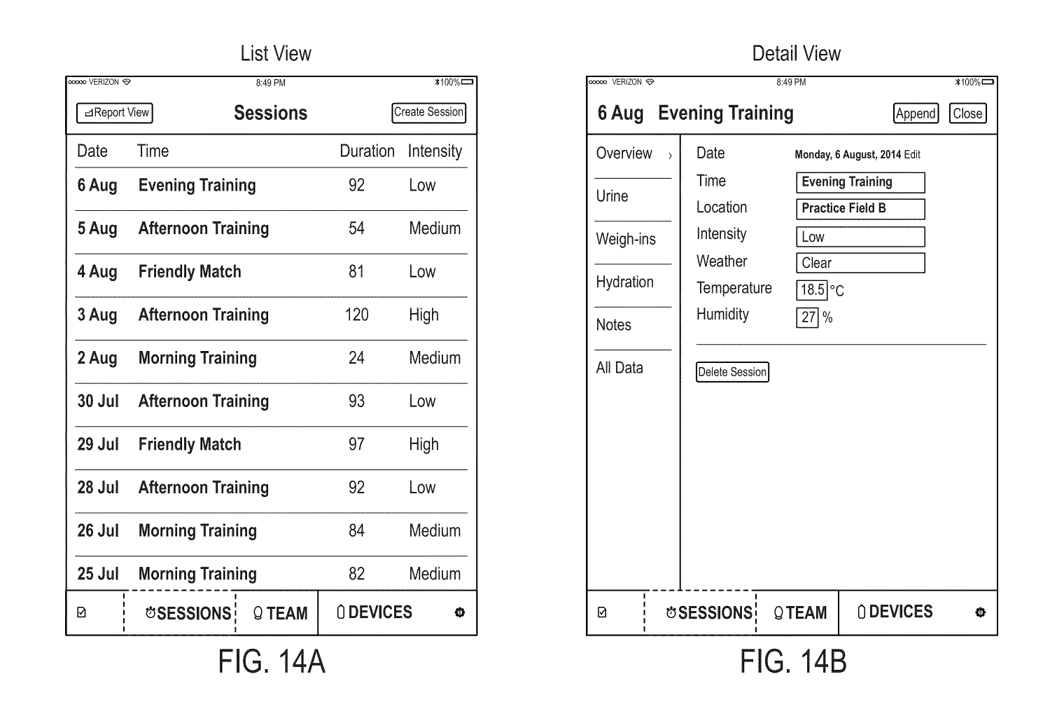

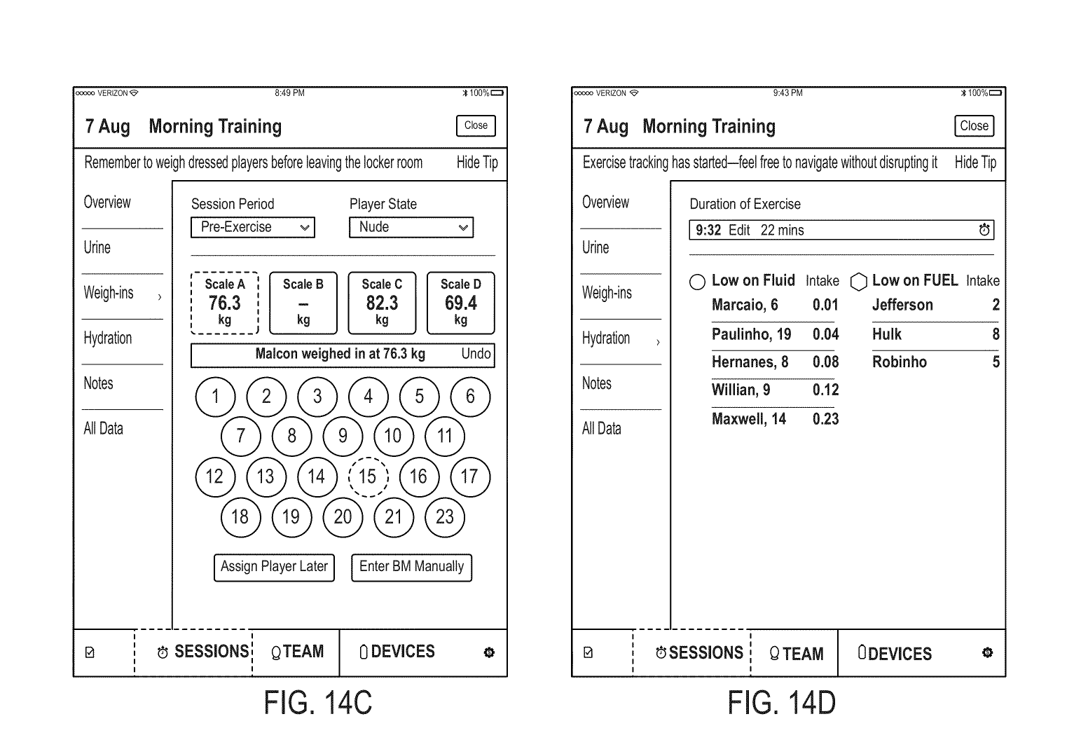

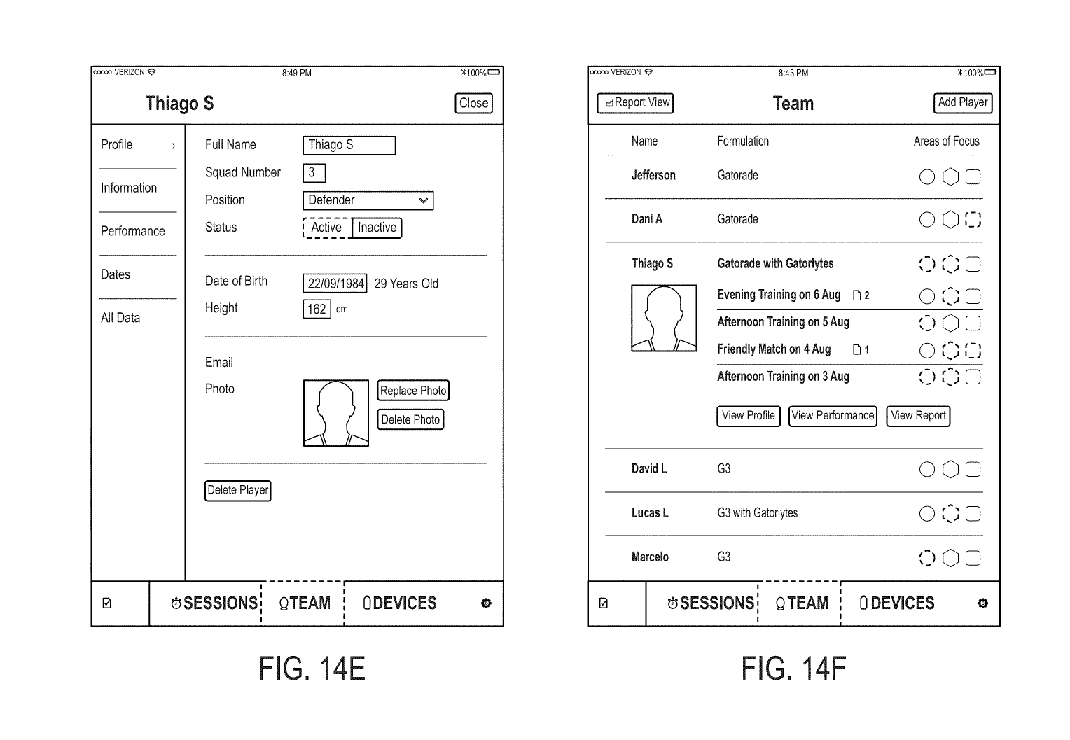

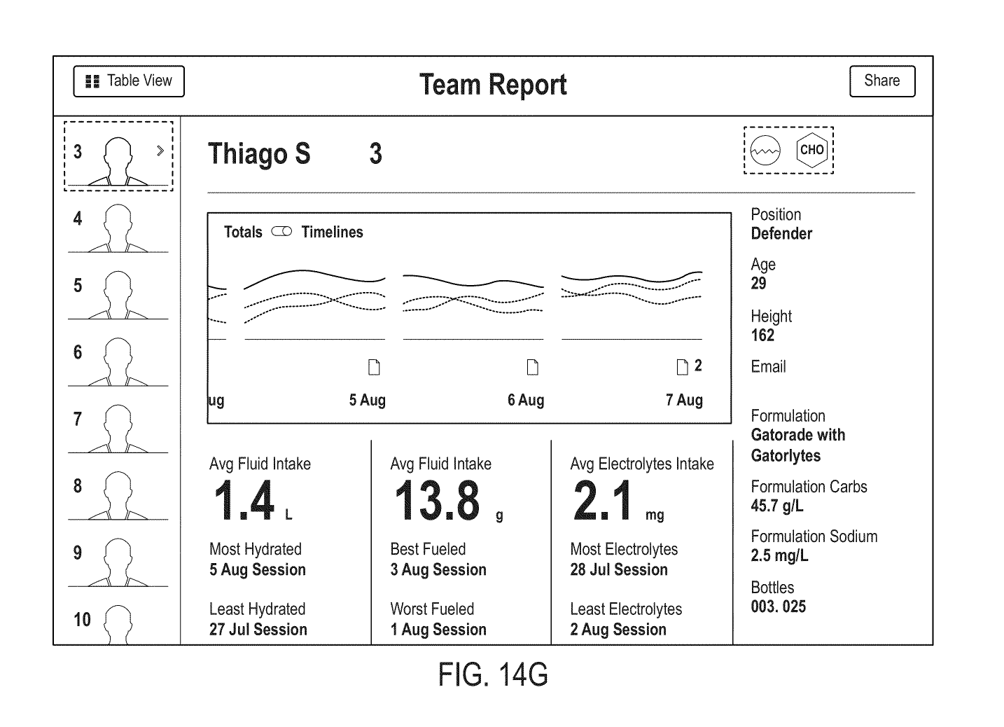

FIG. 14A depicts a list view of multiple sessions. This view may provide dates, activities (e.g. training,) durations of activities, (e.g. 92 minutes) and intensities of the activities (e.g. low, medium, high.)

FIG. 14B depicts a detail view of one of the sessions listed in FIG. 14A. This view may provide the date, activity, location of activity, intensity, weather, temperature, and humidity.

FIG. 14C depicts a weigh-in view and may include activity (e.g. morning training,) when the weigh in occurred (e.g. pre-exercise), player state (nude, clothed), Scale used (e.g. A, B, C, D), weight, and number assigned to the athlete (player.)

FIG. 14D depicts real time analysis and may include activity (e.g. morning training,) duration of exercise, intake of fluids, advisories or warnings if low on fluid intake.

FIG. 14E depicts an athlete (player) detail view and may include name, squad number (or other identifier), position, status (active or inactive), date of birth, height, email, and photo.

FIG. 14F depicts a team detail view and may include a list of athletes (players) and details regarding particular athletes such as fluid being consumed, training periods, and details concerning hydration.

FIG. 14G depicts a report view providing graphics of, for example, fluid, fuel, or electrolytes intake along with other details such as position, age, height, email, formulation consumed (e.g. GATORADE®), and carbohydrates and electrolytes (sodium) in the formulation.

While the invention has been described with respect to specific examples including presently preferred modes of carrying out the invention, those skilled in the art will appreciate that there are numerous variations and permutations of the above described systems and techniques that fall within the spirit and scope of the invention as set forth in the appended claims.

What is claimed is:

1. A hydration monitoring system comprising:
a bottle, wherein the bottle is configured to measure an amount of fluid consumed by an athlete and wirelessly transmit data corresponding to the measurement;
a fluid-loss device, wherein the fluid-loss device is configured to determine an amount of fluid lost by the athlete and wirelessly transmit data corresponding to the amount;
a data communications hub, wherein the hub is configured to receive data from the bottle and the fluid-loss device;
a computer, wherein the computer is configured to receive the data from the hub and determine whether the athlete should consume more fluid based on the data; and
a display for displaying an output conveying the data and the determination.

2. The system of claim 1, wherein the fluid-loss device is a scale configured to measure the athlete's weight.

3. The system of claim 1, wherein the fluid-loss device comprises an input device and a processor, wherein the processor is configured to calculate the amount of fluid lost by the athlete based on a value inputted with the input device.

4. The system of claim 1, wherein the fluid-loss device comprises a sensor.

5. The system of claim 4, wherein the sensor is configured to be wearable by the athlete.

6. The system of claim 5, wherein the sensor includes a porous membrane configured to adhere to the athlete's skin, a microfluidics layer in contact with the porous membrane, a sensor module fluidly connected to the porous membrane, a memory, an antenna, and an outer textile layer.

7. The system of claim 6, wherein the microfluidics layer transports the fluid from the porous membrane to the sensor module, and the sensor determines a change of an ion concentration present in the fluid.

8. The system of claim 5, wherein the sensor includes a layer with a fluid-dependent characteristic.

9. The system of claim 8, wherein the characteristic is impedance or width.

10. The system of claim 4, wherein the sensor is carried by the athlete.

11. The system of claim 10, wherein the sensor comprises a temperature sensor, a moisture sensor, or a pressure sensor.

12. The system of claim 11, wherein the sensor comprises a pedometer.

13. The system of claim 1, wherein the bottle further comprises a display and at least part of the analysis from the computer is transmitted to the display on the bottle.

14. The system of claim 13, wherein the part of the analysis transmitted to the bottle provides an indication whether additional fluid should be consumed.

15. The system of claim 1, wherein the computer is portable.

16. The system of claim 1, wherein the bottle comprises a fluid measurement device to measure the amount of fluid consumed by the athlete, wherein the fluid measurement device is configured to measure the flow of the fluid.

17. The system of claim 16, wherein the fluid measurement device is an Archimedes screw turbine, a thermal mass flow meter, or two wires comprised of resistive material, wherein one wire is shielded from the flow and the other is exposed to the full flow of the fluid.

18. The bottle of claim 17, wherein the Archimedes screw turbine is mounted on hemispherical spigots in a two-part mounting cage.

19. A bottle comprising:
a body;
a removable cap assembly, the cap assembly having
an opening,
a flow meter positioned within the cap assembly below the opening,
electronics to record flow measurements from the flow meter, and
a transceiver to transmit the measurements.

20. The bottle of claim 19, wherein the flow meter is an Archimedes screw turbine, a thermal mass flow meter, or two wires comprised of resistive material, wherein one wire is shielded from the flow and the other is exposed to the full flow of the fluid.

21. The bottle of claim 19, wherein the Archimedes screw turbine is mounted on hemispherical spigots in a two-part mounting cage.

22. The bottle of claim 19, further comprising an LED display.

23. A method of monitoring hydration of an athlete, the method comprising:
measuring an amount of fluid consumed by an athlete from a bottle and periodically transmitting data corresponding to the measurement to a data communications hub;
determining an amount of fluid lost by the athlete using a fluid-loss device and transmitting data corresponding to the amount to the data communications hub;
forwarding the data collected by the data communications hub to a computer; and
determining whether the athlete should consume more fluid based on an analysis of the data; and
displaying the results of the determination on a display.

24. The method of claim 23, further comprising wirelessly transmitting the data from the bottle to the data communications hub, wirelessly transmitting the data from the fluid-loss device to the data communications hub, and wirelessly transmitting the results of the determination to the bottle, and displaying the results of the determination on the bottle.

25. The method of claim 23, further comprising displaying a recommendation to the athlete to consume more or less fluid.

26. The system of claim 13, wherein the display on the bottle is an LED display.
